(19) United States
(12) Patent Application Publication
(10) Pub. No.: US 2023/0071463 A1
(43) Pub. Date: Mar. 9, 2023

(54) SOLID STATE FORMS OF AVASOPASEM MANGANESE AND PROCESS FOR PREPARATION THEREOF

(71) Applicant: TEVA PHARMACEUTICALS INTERNATIONAL GMBH, Jona (CH)

(72) Inventors: Anantha Rajmohan Muthusamy, Sivakasi (IN); Amit Singh, Greater Noida (IN); Yogesh Dhananjay Wagh, Badlapur (E) Thane (IN)

(21) Appl. No.: 17/798,393

(22) PCT Filed: Feb. 12, 2021
(86) PCT No.: PCT/US21/17747

§ 371 Date: Aug. 9, 2022

(30) Foreign Application Priority Data
Feb. 13, 2020 (IN) 202011006283
Mar. 19, 2020 (IN) 202011011821
Jul. 9, 2020 (IN) 202011029249
Nov. 17, 2020 (IN) 202011050050

(51) Int. Cl. C07F 13/00

(52) U.S. Cl. CPC C07F 13/005; C07B 2200/13

(57) ABSTRACT

The present disclosure encompasses solid state forms of Avasopasem manganese, in embodiments crystalline polymorphs of Avasopasem manganese, processes for preparation thereof, and pharmaceutical compositions thereof.

### FIELD OF THE DISCLOSURE

The present disclosure encompasses solid state forms of Avasopasem manganese, in embodiments crystalline polymorphs of Avasopasem manganese, processes for preparation thereof, and pharmaceutical compositions thereof.

### BACKGROUND OF THE DISCLOSURE

Avasopasem manganese (GC4419), has the following chemical structure:

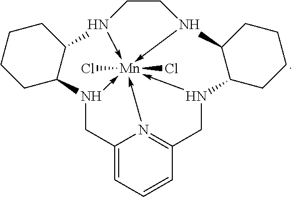

Avasopasem manganese is a highly selective small molecule superoxide dismutase (SOD) mimetic which is being developed for the reduction of radiation-induced severe oral mucositis (SOM). The compound is described in U.S. Pat. No. 8,263,568.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Avasopasem manganese.

### SUMMARY OF THE DISCLOSURE

The present disclosure provides crystalline polymorphs of Avasopasem manganese, processes for preparation thereof, and pharmaceutical compositions thereof. These crystalline polymorphs can be used to prepare other solid state forms of Avasopasem manganese, Avasopasem manganese salts and their solid state forms.

The present disclosure also provides uses of the said solid state forms of Avasopasem manganese in the preparation of other solid state forms of Avasopasem manganese or salts thereof.

The present disclosure provides crystalline polymorphs of Avasopasem manganese for use in medicine, including for the treatment of radiation-induced severe oral mucositis (SOM).

The present disclosure also encompasses the use of crystalline polymorphs of Avasopasem manganese of the present disclosure for the preparation of pharmaceutical compositions and/or formulations.

In another aspect, the present disclosure provides pharmaceutical compositions comprising crystalline polymorphs of Avasopasem manganese according to the present disclosure.

The present disclosure includes processes for preparing the above mentioned pharmaceutical compositions. The processes include combining any one or a combination of the crystalline polymorphs of Avasopasem manganese with at least one pharmaceutically acceptable excipient.

The crystalline polymorph of Avasopasem manganese as defined herein and the pharmaceutical compositions or formulations of the crystalline polymorph of Avasopasem manganese may be used as medicaments, such as for the treatment of radiation-induced severe oral mucositis. Preferably the crystalline forms of Avasopasem manganese as defined herein and the pharmaceutical compositions or formulations of the crystalline polymorph of Avasopasem manganese may be used for the treatment of radiation-induced severe oral mucositis associated with head or neck cancer.

The present disclosure also provides methods of treating radiation-induced severe oral mucositis, by administering a therapeutically effective amount of any one or a combination of the crystalline polymorphs of Avasopasem manganese of the present disclosure, or at least one of the above pharmaceutical compositions, to a subject suffering from radiation-induced severe oral mucositis, or otherwise in need of the treatment. Preferably, the present disclosure also provides methods of treating radiation-induced severe oral mucositis associated with head or neck cancer, by administering a therapeutically effective amount of any one or a combination of the crystalline polymorphs of Avasopasem manganese of the present disclosure, or at least one of the above pharmaceutical compositions, to a subject suffering from radiation-induced severe oral mucositis, or otherwise in need of the treatment.

The present disclosure also provides uses of crystalline polymorphs of Avasopasem manganese of the present disclosure, or at least one of the above pharmaceutical compositions, for the manufacture of medicaments for treating e.g., radiation-induced severe oral mucositis.

### BRIEF DESCRIPTION OF THE DRAWINGS

FIG. 1 shows a characteristic X-ray powder diffraction pattern (XRPD) of Avasopasem manganese Form AM1;

FIG. 2 shows a characteristic XRPD of Avasopasem manganese Form AM2.

FIG. 3 shows a characteristic XRPD of Avasopasem manganese Form AM3.

FIG. 4 shows a characteristic XRPD of Avasopasem manganese Form AM4.

FIG. 5 shows a characteristic XRPD of Avasopasem manganese Form AM5

FIG. 6 shows a characteristic XRPD of Avasopasem manganese Form AM6.

FIG. 7 shows a characteristic XRPD of amorphous Avasopasem manganese.

### DETAILED DESCRIPTION OF THE DISCLOSURE

The present disclosure encompasses solid state forms of Avasopasem manganese, including crystalline polymorphs of Avasopasem manganese, processes for preparation thereof, and pharmaceutical compositions thereof.

Solid state properties of Avasopasem manganese and crystalline polymorphs thereof can be influenced by controlling the conditions under which Avasopasem manganese and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Avasopasem manganese described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Avasopasem manganese. In some embodiments of the disclosure, the described crystalline polymorphs of Avasopasem manganese may contain from about 1% to about 20% (w/w), from about 5% to about 20% (w/w), or from about 5% to about 10% (w/w) of one or more other crystalline polymorph of the same Avasopasem manganese

Depending on which other crystalline polymorphs a comparison is made, the crystalline polymorphs of Avasopasem manganese of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Avasopasem manganese referred to herein as being characterized by graphical data “as depicted in” or “as substantially depicted in” a Figure will thus be understood to include any crystal forms of Avasopasem manganese characterized with the graphical data having such small variations, as are well known to the skilled person, in comparison with the Figure.

As used herein, and unless stated otherwise, the term “anhydrous” in relation to crystalline forms of Avasopasem manganese, relates to a crystalline form of Avasopasem manganese which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Avasopasem manganese of the present disclosure corresponds to a crystalline polymorph of Avasopasem manganese that is physically separated from the reaction mixture in which it is formed.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

As used herein, unless stated otherwise, water content may be determined by thermogravimetric analysis (TGA), preferably at a heating rate of 10° C. per min. More preferably the heating is conducted under nitrogen at a heating rate of 10° C. per min up to 350° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

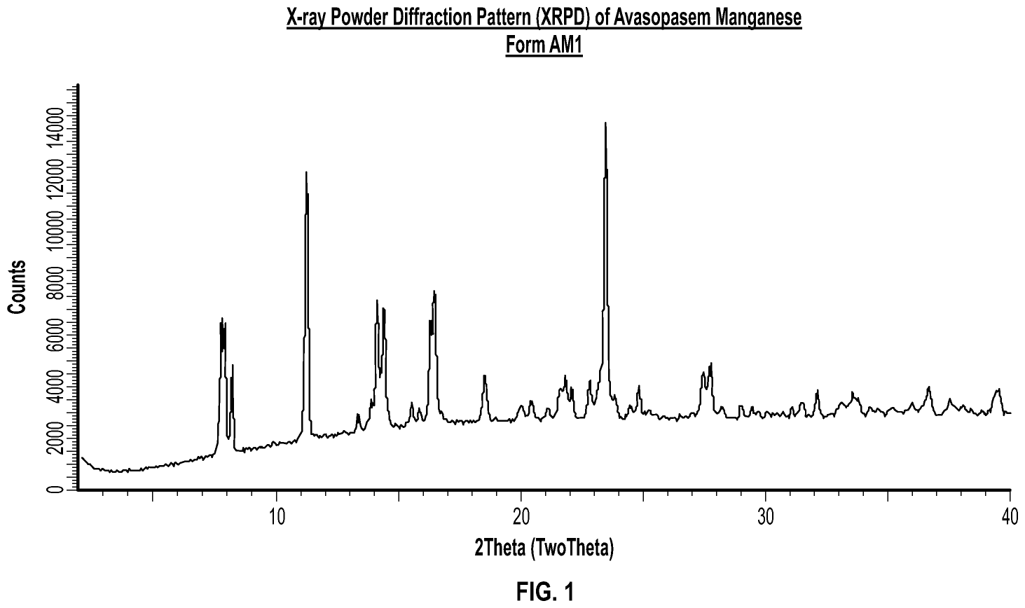

The present disclosure includes a crystalline polymorph of Avasopasem manganese, designated AM1. The crystalline Form AM1 of Avasopasem manganese may be characterized by data selected from one or more of the following: an X-ray powder diffraction pattern substantially as depicted in FIG. 1; an X-ray powder diffraction pattern having peaks at 7.8, 8.2, 11.2, 14.1 and 23.5 degrees 2-theta±0.2 degrees 2-theta; and combinations of these data.

Crystalline Form AM1 of Avasopasem manganese may be further characterized by an X-ray powder diffraction pattern having peaks at 7.8, 8.2, 11.2, 14.1 and 23.5 degrees 2-theta±0.2 degrees 2-theta, and also having any one, two, three, four or five additional peaks selected from 16.5, 18.5, 24.8, 27.7 and 32.1 degrees 2-theta±0.2 degrees 2-theta.

Crystalline Form AM1 of Avasopasem manganese may be alternatively characterized by an X-ray powder diffraction pattern having peaks at 7.8, 8.2, 11.2, 14.1, 16.5, 18.5, 23.5, 24.8, 27.7 and 32.1 degrees 2-theta±0.2 degrees 2-theta.

In one embodiment of the present disclosure, crystalline Form AM1 of Avasopasem manganese is isolated.

Crystalline Form AM1 of Avasopasem manganese may be a hydrate; more preferably monohydrate. Preferred percentage range for water in the hydrate is 3-5.5%. Alternatively, crystalline form AM1 is a hydrated form comprising about 3 wt % to about 5.5 wt % water, or about 3 wt % to about 5 wt % water. In any embodiment, Form AM1 may comprise about 3.2 wt % to about 4.8 wt % water, and optionally about 3.3 wt % to about 3.8 wt % water. In embodiments

Form AM1 of Avasopasem manganese according to the present invention is stable when exposed to high relative humidity.

The present invention also provides a process for preparing Form AM1 of Avasopasem manganese. In embodiments, the process may comprise removal of solvent from a solution of Avasopasem manganese to form crystalline form AM1 of Avasopasem manganese.

In any embodiment, the process may comprise:

(i) combining Avasopasem manganese and a solvent, optionally with heating, to form a solution;
(ii) optionally filtering the solution;
(iii) evaporating the solvent from the solution to provide Avasopasem manganese Form AM1; and
(iv) optionally isolating and/or drying the Form AM1.

In any embodiment of the process, the solvent is preferably selected from an alcohol (preferably isopropanol, 1-propanol, 1-butanol, or ethanol), dichloromethane, isopropyl acetate, or water. More preferably, the solvent is selected from isopropanol, dichloromethane, or water. In any embodiment, the solvent removal may be carried out by evaporation, for example by exposure to air, or by heating, or by heating under reduced pressure. When the solvent is other than water, the process may be carried out in the presence of trace amounts of water, for example trace amounts of water may be present in the solvent, or the process may be carried out via exposure to ambient relative humidity conditions, such as by conducting the solvent removal by exposure of the solution to room temperature and ambient relative humidity.

Preferably, step (i) comprises dissolving Avasopasem manganese in a solvent selected from the group consisting of an alcohol (preferably isopropanol, ethanol, 1-propanol or 1-butanol), isopropyl acetate, dichloromethane or water. The Avasopasem manganese may be combined with the solvent to form a solution. The solution may be formed by dissolving Avasopasem in a solvent which may be optionally heated, or by combining Avasopasem and a solvent and optionally heating to form a solution. The heating of the solvent or of the solution may be carried out to a temperature of up to the reflux temperature of the solvent or mixture. For example, the temperature may be at about 25° C. to about 80° C., about 25° C. to about 70° C. The volume of solvent to Avasopasem manganese may be: about 2 ml to about 40 ml, about 4 ml to about 30 ml, or about 4.5 ml to about 29 ml per gram of Avasopasem manganese. Preferably step (ii) may comprise filtering the solution, optionally to remove insoluble matter. In any embodiment step (iii) may comprise evaporating the solvent from the solution to form crystalline Form AM1. The evaporation may be carried out by exposure of the solution to the air at ambient conditions of temperature and relative humidity. Preferably when the solvent is dichloromethane, step (iii) is conducted by slow evaporation of the solvent by exposure of the solution to the atmosphere. Alternatively, the evaporation of the solvent may be conducted by distillation at elevated temperature, and optionally at reduced pressure. Suitable temperatures may be up to the reflux temperature of the solution, for example about 40° C. to about 80° C., about 50° C. to about 75° C., or about 60° C. to about 70° C., or about 65° C. Preferably this distillation step is carried out when the solvent is an alcohol or water. Following removal of the solvent, the crystalline Form AM1 may be isolated. In any embodiment, step (iv) comprises drying the crystalline Form AM1 under reduced pressure, preferably at a temperature of about 25° C. to about 80° C., about 45° C. to about 70° C., or about 55° C. to about 70° C., or about 65° C. The drying may be conducted for a period of about 10 to about 60 minutes, about 15 to about 45 minutes, about 20 to about 40 minutes, or about 30 minutes.

In a further aspect, the invention provides a process for preparing Form AM1 of Avasopasem manganese, comprising cooling a solution of Avasopasem manganese in a solvent. The process may comprise:

(i) providing a mixture of Avasopasem manganese in an organic solvent selected from: alcohol (preferably wherein the alcohol is selected from: ethanol; isopropyl alcohol, 2-butanol, t-butyl alcohol, cyclopentanol, 1-pentanol, 2-methoxyethanol, 2-ethoxyethanol, tert-amyl alcohol and iso-amyl alcohol) and carboxylic acid (preferably, formic acid, acetic acid and propionic acid);
(ii) heating the mixture;
(iii) cooling;
(iv) optionally adding an antisolvent;
(v) stirring the mixture;
(vi) optionally isolating the Form AM1 of Avasopasem; and
(vii) optionally isolating and/or drying the Form AM1 of Avasopasem.

In any embodiment of this process, step (i) may comprise combining Avasopasem manganese with the organic solvent. Preferably the solvent is selected from C₂ to C₆ alcohols, more preferably ethanol; isopropyl alcohol, 2-butanol, and t-butyl alcohol; and more preferably ethanol). Preferably, the volume of solvent to Avasopasem manganese may be: about 2 ml to about 10 ml, about 3 ml to about 8 ml, or about 4 ml to about 6 ml, or about 5 ml, per gram of Avasopasem manganese. In any embodiment of this process, step (ii) may comprise heating the mixture to a temperature of up to the reflux temperature, preferably wherein the temperature is about 40° C. to about 80° C., about 50° C. to about 70° C., about 60° C. to about 70° C. Form AM1 may be obtained by cooling the mixture to room temperature, and optionally stirring for a suitable period of time. In any embodiment, the cooling may be to about 5° C. to about 40° C., about 15° C. to about 30° C., about 20° C. to about 28° C. In any embodiment, step (c) includes a step of stirring the mixture for a suitable period of time, preferably about be conducted for a period of about 8 to about 48 hours, about 15 to about 30 hours, or about 18 to about 25 hours. In any embodiment of this process, step (iii) may comprise cooling the mixture to a temperature of about 10° C. to about 35° C., about 15° C. to about 30° C., about 20° C. to about 28° C., or about 25° C. The cooling may be carried over a period of about 15 minutes to about 200 minutes, about 30 to about 100 minutes, about 30 to about 80 minutes, about 35 to about 60 minutes, about 40 to about 50 minutes, or about 45 minutes. After the cooling step, an antisolvent may be added to the mixture [step (iv)]. Suitable antisolvents include ethers or ketones, preferably C₄-C₈ ethers, C₄-C₈ diethers, or C₃-C₈ ketones. Preferably the antisolvent is selected from methyl tert-butyl ether, 1,2-dimethoxyethane, 2-methyl tetrahydrofuran, methyl isobutyl ketone, ethyl acetate and methyl ethyl ketone. The antisolvent may be added in a ratio of about 0.5 ml to about 10 ml, about 1 ml o about 5 ml, or about 1 ml to about 3 ml, or about 2.5 ml, per ml of solvent. In any embodiment of the process, step (v) comprises stirring the mixture. The mixture may be stirred at a temperature of: about 10° C. to about 35° C., about 15° C. to about 30° C., about 20° C. to about 28° C., or about 25° C. The stirring may be carried out over a period of: about 8 to about 48 hours, about 10 to about 30 hours, or about 16 to about 26 hours. In any embodiment of this process, Avasopasem manganese Form AM1 may be isolated by filtration or decantation, preferably by suction filtration. The product may be dried by suction filtration, for example for about 2 to about 20 minutes, about 2 to about 10 minutes, or about 5 minutes. In any embodiment of this process, the process may be carried out in the presence of trace amounts of water, for example trace amounts of water may be present in the solvent, or the process may be carried out via exposure to ambient relative humidity conditions.

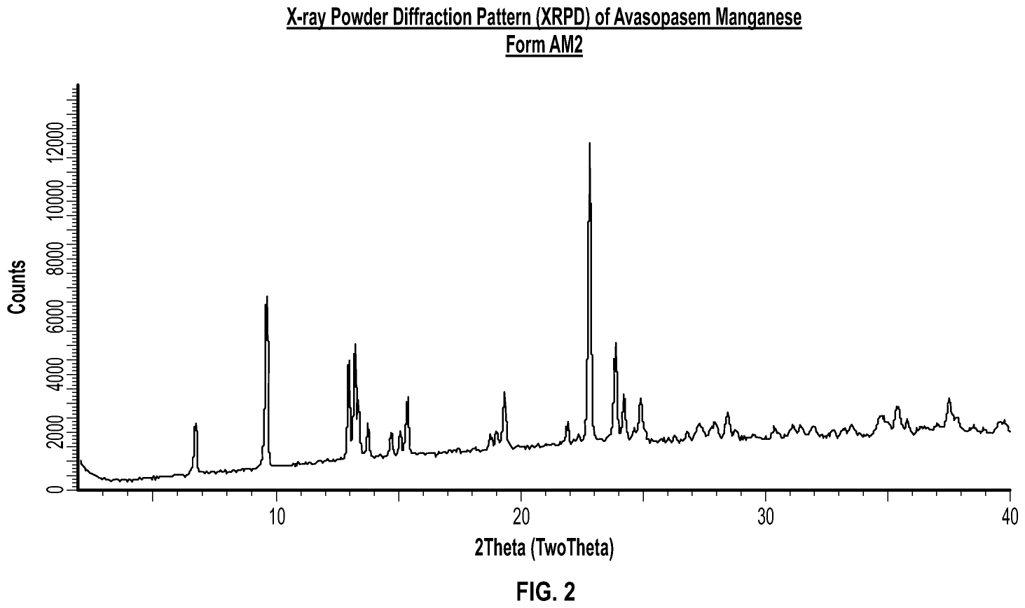

In a further embodiment, the present disclosure encompasses a crystalline polymorph of Avasopasem manganese, designated AM2. The crystalline Form AM2 of Avasopasem manganese may be characterized by data selected from one or more of the following: an X-ray powder diffraction pattern substantially as depicted in FIG. 2; an X-ray powder diffraction pattern having peaks at 6.8, 9.7, 13.0, 22.9 and 23.9 degrees 2-theta±0.2 degrees 2-theta; and combinations of these data.

Crystalline Form AM2 of Avasopasem manganese may be further characterized by an X-ray powder diffraction pattern having peaks at 6.8, 9.7, 13.0, 22.9 and 23.9 degrees 2-theta±0.2 degrees 2-theta, and also having any one, two, three, four or five additional peaks selected from 13.3, 15.4, 19.4, 24.3 and 24.9 degrees 2-theta±0.2 degrees 2-theta.

Crystalline Form AM2 of Avasopasem manganese may be alternatively characterized by an X-ray powder diffraction pattern having peaks at 6.8, 9.7, 13.0, 13.3, 15.4, 19.4, 22.9, 23.9, 24.3 and 24.9 degrees 2-theta±0.2 degrees 2-theta.

In one embodiment of the present disclosure, crystalline Form AM2 of Avasopasem manganese is isolated.

Crystalline Form AM2 of Avasopasem manganese may be a hydrate; more preferably dihydrate. Preferred percentage range for water in the hydrate is 5-9%. Preferably crystalline form AM2 of Avasopasem manganese is a hydrated form comprising about 5 wt % to about 9 wt % water. In any embodiment Form AM2 may be a hydrate comprising about 5.8 wt % to about 8.5 wt % water, or about 6 wt % to about 7.5 wt % water. In any embodiment, Form AM2 may comprise about 6.5 wt % to about 7 wt % water.

Crystalline Form AM2 of Avasopasem manganese is stable when exposed to high temperatures and high relative humidity.

The present invention also provides a process for obtaining Form AM2 of Avasopasem manganese, comprising stirring a suspension of Avasopasem manganese in water. In any embodiment, the process can comprise:

(i) providing a suspension of Avasopasem manganese in water;
(ii) stirring the suspension;
(iii) isolating Avasopasem manganese Form AM2 from the mixture; and
(iv) optionally drying the product.

In any embodiment of this process, the suspension may comprise water in an amount of about 1 ml to about 10 ml, about 2 ml to about 8 ml, about 3 ml to about 7 ml, about 4 ml to about 6 ml, or about 5 ml per gram of Avasopasem manganese. Preferably the suspension is at a temperature of: about 0° C. to about 35° C., about 0° C. to about 30° C., or about 0° C. to about 28° C. In any embodiment of the process, step (ii) can comprise stirring the suspension at this temperature for about 8 to about 48 hours, about 10 to about 30 hours, or about 16 to about 26 hours, or about 24 hours. In any embodiment of this process, step (iii) may comprise filtering or decantation, preferably step (iii) comprises suction filtration. The product may be dried by suction filtration, for example for about 2 to about 20 minutes, about 2 to about 10 minutes, or about 5 minutes.

The present invention also provides a process for obtaining Form AM2 of Avasopasem manganese, comprising cooling a solution of Avasopasem manganese in a solvent comprising water. In any embodiment this process may comprise:

(i) providing a mixture of Avasopasem manganese in water;
(ii) heating the mixture to form a solution;
(iii) cooling;
(iv) optionally adding an antisolvent;
(v) stirring the mixture;
(vi) optionally isolating the Form AM2 of Avasopasem manganese; and
(vii) optionally drying the Form AM2 of Avasopasem manganese.

Step (i) may comprise combining Avasopasem manganese with water. The water may be present in an amount of: about 10 ml to about 30 ml, about 12 ml to about 25 ml, about 15 ml to about 22 ml, about 16 ml to about 20 ml, or about 18 ml per gram of Avasopasem manganese. Preferably the suspension is heated to a temperature of: about 40° C. to about 90° C., about 45° C. to about 80° C., or about 50° C. to about 75° C., or about 55° C. to about 70° C., or about 60° C. to about 65° C. In any embodiment of the process, step (iii) can comprise cooling the mixture to a temperature of: about 10° C. to about 35° C., about 15° C. to about 30° C., about 20° C. to about 28° C., or about 25° C. In any embodiment of the process, an antisolvent may be added to the mixture [step (iv)]. The antisolvent may comprise a water-miscible ether, preferably a C₄-C₈ ether or a C₄-C₈ diether, more preferably a C₄-C₈ diether, and particularly 1,2-dimethoxyethane. The antisolvent may be added in a ratio of about 0.5 ml to about 10 ml, about 1 ml to about 8 ml, about 1 ml to about 5 ml, about 2 ml, to about 4 ml, or about 3 ml, per ml of solvent. In any embodiment of this process, step (v) comprises stirring the mixture. The mixture may be stirred at a temperature of: about 0° C. to about 25° C., about 0° C. to about 15° C., about 0° C. to about 10° C., or about 0° C. to about 5° C. The stirring may be carried out over a period of: about 1 to about 10 hours, about 2 to about 8 hours, about 3 to about 6 hours, or about 4 hours. In any embodiment of this process, Avasopasem manganese Form AM2 may be isolated by filtration or decantation, preferably by suction filtration. The product may be dried by suction filtration, for example for about 2 to about 20 minutes, about 2 to about 10 minutes, or about 5 minutes.

The present invention provides a further process for preparing Form AM2 of Avasopasem manganese. In embodiments, the process may comprise removal of solvent from a solution of Avasopasem manganese in a solvent comprising water, to form crystalline form AM2 of Avasopasem manganese. In any embodiment, the process may comprise:

(i) combining Avasopasem manganese and a solvent comprising water, optionally with heating, to form a solution;
(ii) optionally filtering the solution;
(iii) evaporating the solvent from the solution to provide Avasopasem manganese Form AM2; and
(iv) optionally isolating and/or drying the Form AM2 Avasopasem manganese.

In any embodiment of this process, the solvent is preferably selected from a mixture of water and a water-miscible ether, preferably a C₄-C₈ ether or a C₄-C₈ diether, more preferably a C₄-C₈ cyclic ether, particularly tetrahydrofuran, or 2-methyltetrahydrofuran and most preferably tetrahydrofuran. Water may be present in a ratio of: about 0.5 ml to about 5 ml, about 0.7 ml to about 4 ml, about 1 ml to about 3 ml, about 1 ml to about 2 ml, or about 1 ml to about 1.5 ml, or about 1.3 ml per ml of solvent. Water may be present in a ratio of: about 1 ml to about 10 ml, about 1 ml to about 8 ml, about 2 ml to about 6 ml, about 3 ml to about 5 ml, or about 4 ml per gram of Avasopasem manganese. In any embodiment, the mixture in step (i) comprise Avasopasem manganese, water and THF. The mixture in step (i) may be at a temperature of: about 20° C. to about 45° C., about 20° C. to about 35° C., or about 25° C. to about 30° C. Preferably step (ii) comprises filtering the solution, optionally to remove insoluble matter. In any embodiment step (iii) may comprise evaporating the solvent from the solution to form crystalline Form AM2. The evaporation may be carried out by exposure of the solution to the air at ambient conditions of temperature and relative humidity. Preferably when the solvent is dichloromethane, step (iii) is conducted by slow evaporation of the solvent by exposure of the solution to the atmosphere.

In a further aspect of the present invention, there is provided a process for preparing Form AM2 of Avasopasem manganese comprising:
providing Avasopasem manganese in a solvent selected from: isopropyl alcohol and water.
heating up to reflux temperature (preferably to 65° C.)
isolating Form AM2 by cooling to room temperature and, optionally, by adding an anti-solvent (preferably wherein the anti-solvent is selected from: dimethyl carbonate, hexane and heptane and 1, 2-dimethoxyethane).

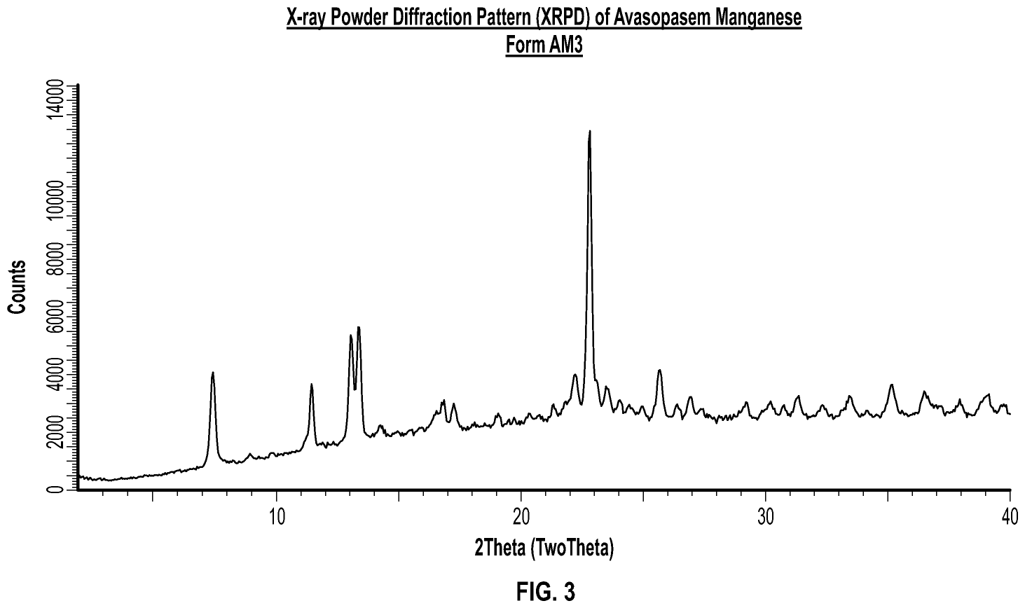

In a further embodiment, the present disclosure includes a crystalline polymorph of Avasopasem manganese, designated AM3. The crystalline Form AM3 of Avasopasem manganese may be characterized by data selected from one or more of the following: an X-ray powder diffraction pattern substantially as depicted in FIG. 3; an X-ray powder diffraction pattern having peaks at 7.4, 11.5, 13.4, 17.3 and 25.7 degrees 2-theta±0.2 degrees 2-theta; and combinations of these data.

Crystalline Form AM3 of Avasopasem manganese may be further characterized by an X-ray powder diffraction pattern having peaks at 7.4, 11.5, 13.4, 17.3 and 25.7 degrees 2-theta±0.2 degrees 2-theta, and also having any one, two, three or four additional peaks selected from 13.1, 16.9, 22.2 and 22.8 degrees 2-theta±0.2 degrees 2-theta.

Crystalline Form AM3 of Avasopasem manganese may be alternatively characterized by an X-ray powder diffraction pattern having peaks at 7.4, 11.5, 13.1, 13.4, 16.9, 17.3, 22.2, 22.8 and 25.7 degrees 2-theta±0.2 degrees 2-theta.

In one embodiment of the present disclosure, crystalline Form AM3 of Avasopasem manganese is isolated.

Crystalline Form AM3 of Avasopasem manganese may be a hydrate; more preferably a tetrahydrate. Preferred percentage range for water is 11-15%. Preferably crystalline form AM3 is a hydrate form comprising about 11 wt % to about 15 wt % water. In any embodiment Form AM3 may comprise about 11 wt % to about 14 wt % water, or about 11.5 wt % to about 13.5 wt % water. In any embodiment, Form AM3 may comprise from 11.8 wt % to about 13.2 wt % water.

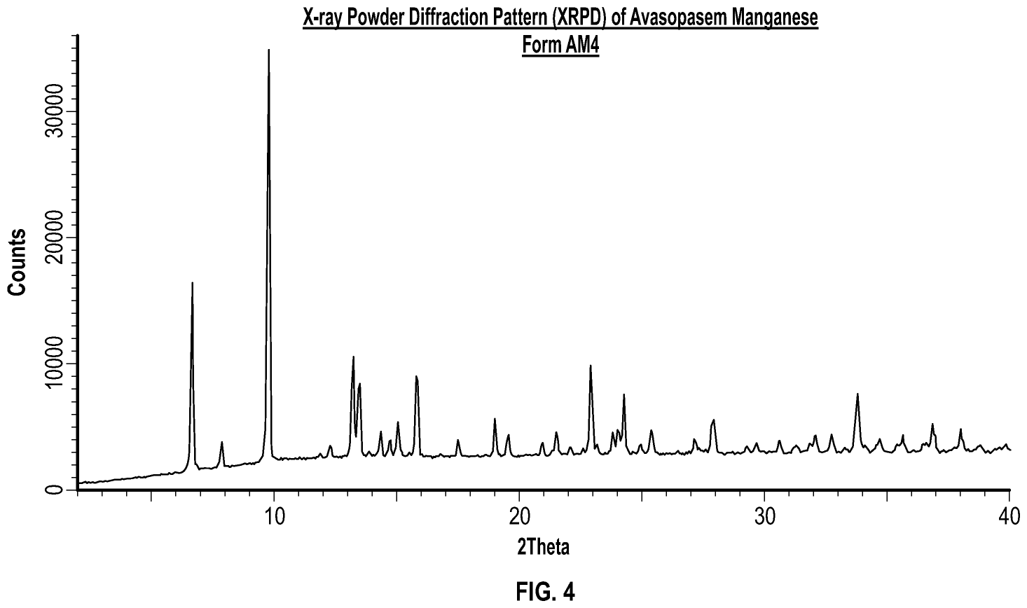

In another embodiment, the present disclosure includes a crystalline polymorph of Avasopasem manganese, designated AM4. The crystalline Form AM4 of Avasopasem manganese may be characterized by data selected from one or more of the following: an X-ray powder diffraction pattern substantially as depicted in FIG. 4; an X-ray powder diffraction pattern having peaks at 7.9, 14.3, 15.8, 25.4 and 28.0 degrees 2-theta±0.2 degrees 2-theta; and combinations of these data.

Crystalline Form AM4 of Avasopasem manganese may be further characterized by an X-ray powder diffraction pattern having peaks at 7.9, 14.3, 15.8, 25.4 and 28.0 degrees 2-theta±0.2 degrees 2-theta, and also having any one, two, three or four additional peaks selected from 6.7, 9.8, 19.0, 21.5 and 33.8 degrees 2-theta±0.2 degrees 2-theta.

Crystalline Form AM4 of Avasopasem manganese may be alternatively characterized by an X-ray powder diffraction pattern having peaks at 6.7, 7.9, 9.8, 14.3, 15.8, 19.0, 21.5, 25.4, 28.0, and 33.8 degrees 2-theta±0.2 degrees 2-theta.

In one embodiment of the present disclosure, crystalline Form AM4 of Avasopasem manganese is isolated.

Crystalline Form AM4 of Avasopasem manganese may be a hydrate; more preferably dihydrate. Preferred percentage range for water is 5 wt % to 9 wt %. In any embodiment Form AM4 may be a hydrate comprising about 5.8 wt % to about 8.5 wt % water, or about 6 wt % to about 7.5 wt % water. In any embodiment, Form AM4 may comprise about 6.5 wt % to about 7 wt % water.

Crystalline Form AM4 of Avasopasem manganese as described herein is stable when exposed to high temperatures and high relative humidity.

The present invention also provides a process for obtaining Form AM4 of Avasopasem manganese. The process comprises stirring a suspension of Avasopasem manganese in a solvent comprising water or in a mixture of organic solvent and water, to obtain Avasopasem manganese Form AM4. In any embodiment, the process comprises:

(i) providing a mixture of Avasopasem manganese in water or a solvent comprising water and an organic solvent, preferably wherein the organic solvent is selected from 1,2,-Dimethoxyethane, isopropyl alcohol, tetrahydrofuran and acetonitrile, to form a suspension;
(ii) heating the mixture;
(v) optionally cooling to obtain Form AM4 of Avasopasem manganese;
(vi) optionally isolating the Form AM4 of Avasopasem manganese; and
(vii) optionally drying the Form AM4 of Avasopasem manganese.

Step (i) may comprise combining Avasopasem manganese with water and optionally the organic solvent. The water may be present in an amount of: 0.1 to about 10 ml, about 0.1 ml to about 8 ml, about 0.2 ml to about 6 ml, about 0.3 ml to about 5 ml per gram of Avasopasem manganese. When water is used alone, the amount of water is: about 1 to about 10 ml, about 2 ml to about 8 ml, about 3 ml to about 6 ml, or about 5 ml per gram of Avasopasem manganese. When a mixture of water and the organic solvent is used, the water may be present in an amount of: 0.1 to about 5 ml, about 0.1 ml to about 4 ml, about 0.2 ml to about 3 ml, about 0.3 ml to about 2.5 ml per gram of Avasopasem manganese. When a mixture of water and the organic solvent is used, the organic solvent may be present in an amount of: about 1 to about 15 ml, about 1 ml to about 12 ml, about 2 ml to about 10 ml, about 2.5 ml to about 9 ml per gram of Avasopasem manganese. The organic solvent may be used in a ratio of: about 1 ml to about 12 ml, about 2 ml to about 10 ml, about 3 ml to about 9 ml, per ml of water.

Preferably the suspension is heated to a temperature of: about 60° C. to about 95° C., about 60° C. to about 90° C., or about 60° C. to about 80° C., or about 60° C. to about 75° C. The mixture may be heated to this temperature for a period of about 8 hours to about 48 hours, about 12 hours to about 36 hours, about 18 hours to about 28 hours, or about 16 hours to about 24 hours. In any embodiment of the process, the mixture may be filtered, preferably by suction filtration, to isolate the product. The mixture may be cooled prior to filtration, typically to a temperature of: about 10° C. to about 35° C., about 15° C. to about 30° C., about 20° C. to about 28° C., or about 25° C. The crystalline Form AM4 of Avasopasem manganese may be dried by suction filtration, for example, for about 2 to about 40 minutes, about 2 to about 30 minutes, or about 5 to about 15 minutes.

The present invention further provides another process for preparing Form AM4, comprising exposure of Avasopasem Manganese (preferably Amorphous Avasopasem manganese) to high humidity. The relative humidity may be from about 60% to about 100%, about 80% to about 100%, about 90% to about 100%, or about 90%. The exposure may be carried out at a temperature of about 40° C. to about 80° C., about 50° C. to about 70° C., about 55° C. to about 65° C., about 58° C. to about 62° C. or about 60° C.

The exposure may be carried out over a period of about 0.5 days to about 7 days, about 1 day to about 5 days, about 2 days to about 4 days, or about 3 days.

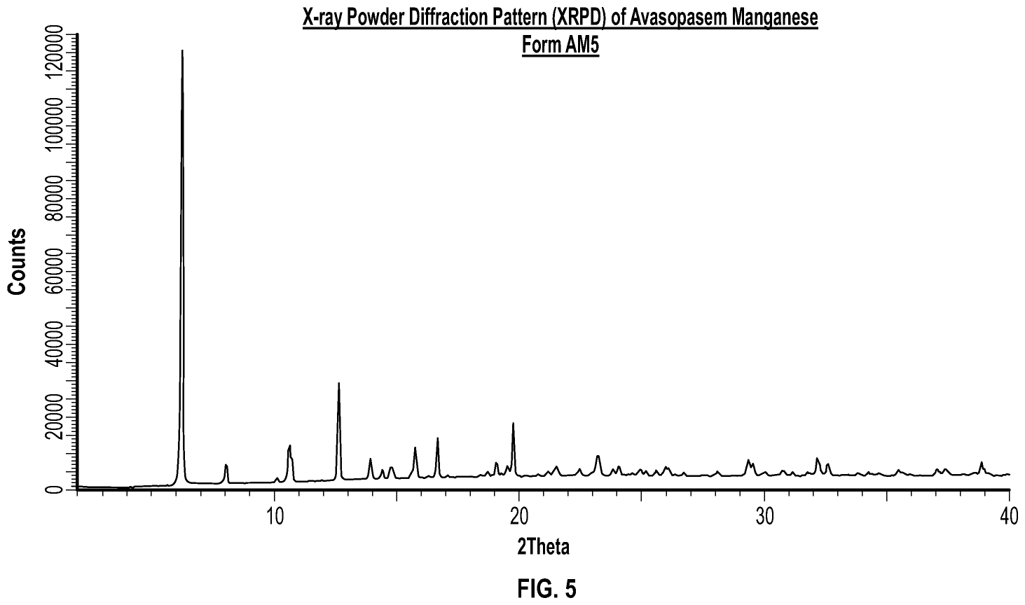

In a further embodiment, the present disclosure includes a crystalline polymorph of Avasopasem manganese, designated AM5. The crystalline Form AM5 of Avasopasem manganese may be characterized by data selected from one or more of the following: an X-ray powder diffraction pattern substantially as depicted in FIG. 5; an X-ray powder diffraction pattern having peaks at 6.3, 10.6, 12.7, 19.8 and 23.2 degrees 2-theta±0.2 degrees 2-theta; and combinations of these data.

Crystalline Form AM5 of Avasopasem manganese may be further characterized by an X-ray powder diffraction pattern having peaks at 6.3, 10.6, 12.7, 19.8 and 23.2 degrees 2-theta±0.2 degrees 2-theta, and also having any one, two or three additional peaks selected from 8.1, 14.0 and 16.7 degrees 2-theta±0.2 degrees 2-theta.

Crystalline Form AM5 of Avasopasem manganese may be further characterized by an X-ray powder diffraction pattern having peaks at 6.3, 8.1, 10.6, 12.7, 14.0, 16.7, 19.8 and 23.2 degrees 2-theta±0.2 degrees 2-theta.

Crystalline Form AM5 of Avasopasem manganese may be aniline solvate.

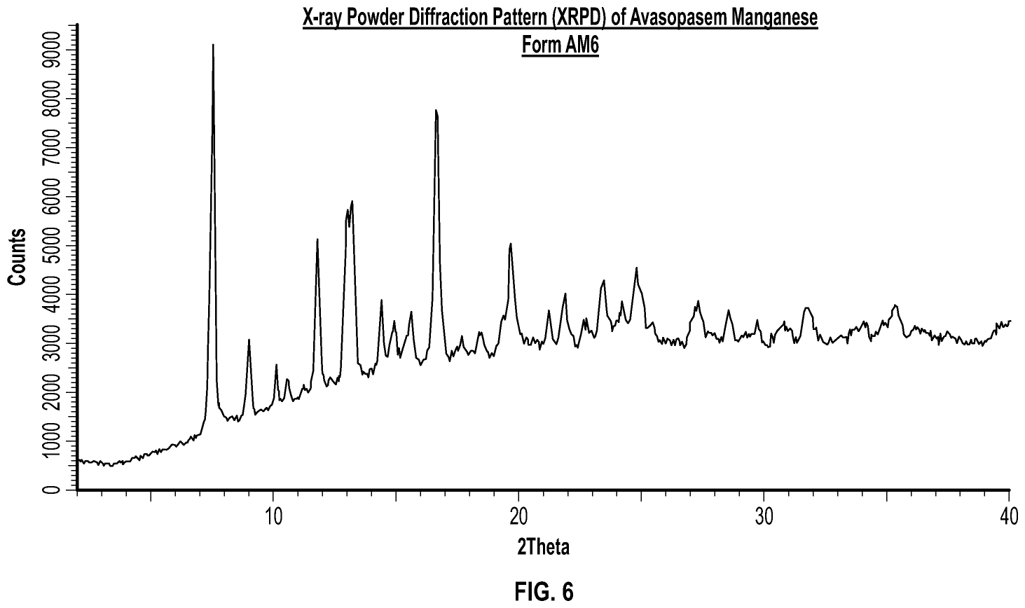

The present disclosure also includes a crystalline polymorph of Avasopasem manganese, designated AM6. The crystalline Form AM6 of Avasopasem manganese may be characterized by data selected from one or more of the following: an X-ray powder diffraction pattern substantially as depicted in FIG. 6; an X-ray powder diffraction pattern having peaks at 7.5, 9.0, 11.8, 16.7 and 19.7 degrees 2-theta±0.2 degrees 2-theta; and combinations of these data.

Crystalline Form AM6 of Avasopasem manganese may be further characterized by an X-ray powder diffraction pattern having peaks at 7.5, 9.0, 11.8, 16.7 and 19.7 degrees 2-theta±0.2 degrees 2-theta, and also having any one, two, three or four additional peaks selected from 10.1, 10.6 and 14.9 degrees 2-theta±0.2 degrees 2-theta.

Crystalline Form AM6 of Avasopasem manganese may be a hydrate; more preferably hemihydrate. Preferred percentage range for water is 1-3%. Preferably crystalline form AM6 is a hydrate form comprising about 1 wt % to about 3 wt % water In any embodiment Form AM6 may be a hydrate comprising about 1.5 wt % to about 2.8 wt % water, or about 1.6 wt % to about 2.5 wt % water. In any embodiment, Form AM6 may comprise about 1.7 wt % to about 2.3 wt % water.

The above crystalline polymorphs can be used to prepare other crystalline polymorphs of Avasopasem manganese, Avasopasem manganese salts and their solid state forms.

The present disclosure encompasses a process for preparing other solid state forms of Avasopasem manganese, Avasopasem manganese salts and their solid state forms thereof. The process includes acidifying any one or a combination of the above described polymorphs of Avasopasem manganese to obtain the corresponding salt.

The present disclosure provides the above described crystalline polymorphs of Avasopasem manganese for use in the preparation of pharmaceutical compositions comprising Avasopasem manganese and/or crystalline polymorphs thereof.

The present disclosure also encompasses the use of crystalline polymorphs of Avasopasem manganese of the present disclosure for the preparation of pharmaceutical compositions of crystalline polymorph Avasopasem manganese and/or crystalline polymorphs thereof.

The present disclosure includes processes for preparing the above mentioned pharmaceutical compositions. The processes include combining any one or a combination of the crystalline polymorphs of Avasopasem manganese of the present disclosure with at least one pharmaceutically acceptable excipient.

Pharmaceutical combinations or formulations of the present disclosure contain any one or a combination of the solid state forms of Avasopasem manganese of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Avasopasem manganese and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Avasopasem manganese can be administered. Avasopasem manganese may be formulated for administration to a mammal, in embodiments to a human, by injection. Avasopasem manganese can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Avasopasem manganese and the pharmaceutical compositions and/or formulations of Avasopasem manganese of the present disclosure can be used as medicaments, in embodiments in the treatment of radiation-induced severe oral mucositis.

The present disclosure also provides methods of treating radiation-induced severe oral mucositis by administering a therapeutically effective amount of any one or a combination of the crystalline polymorphs of Avasopasem manganese of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

X-ray diffraction was performed on X-Ray powder diffractometer: Bruker D8 Advance; CuKα radiation (λ=1.54 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

Measurement parameters:
Scan range: 2-40 degrees 2-theta;
Scan mode: continuous;
Step size: 0.05 degrees;
Time per step: 0.5 s;
Sample spin: 30 rpm;
Sample holder: PMMA specimen holder ring.
All X-Ray Powder Diffraction peak values are calibrated with regard to standard silicon spiking in the sample.

### Thermogravimetric Analysis (TGA″) Method:

Thermogravimetric analysis was conducted on TA instrument TGA Q-500 Thermogravimetric analyzer. About 5-15 mg sample was placed into a tared TGA crucible and placed into a TGA furnace. The furnace was heated under nitrogen at a heating rate of 10° C./min up to 350° C.

### EXAMPLES

### Preparation of Starting Materials

Avasopasem manganese can be prepared according to methods known from the literature, for example U.S. Pat. No. 8,263,568. Alternatively, Avasopasem manganese can be prepared by the template method reported for the enantiomeric analogue GC4403, which has the formula:

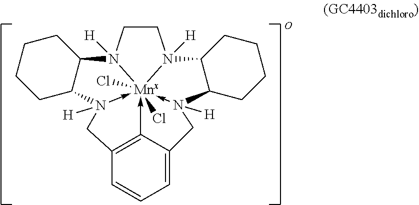

GC4403 is disclosed in International Appl. No. WO 98/58636 (as compound SC-72325) and Riley, D. P, and Schall, O. F., Advances in Inorganic Chemistry (2007), 59, 233-263. Thus, GC4403 can be synthesized via the template route described in the literature using the chiral R,R-1,2-diamminocyclohexane [Salvemini, D., et al., Science (1999), 286, 304-6, and Aston, K, et al., Inorg. Chem. (2001), 40(8), 1779-89]. Avasopasem manganese (GC4419) can be prepared by the same method except that the chiral R,R-1,2-diamminocyclohexane is replaced with S,S-1,2-diamminocyclohexane.

### Example 1: Preparation of Avasopasem Manganese Form AM1

Avasopasem manganese (0.1 grams) was dissolved in dichloromethane (0.5 ml) at 25-30° C. in a test tube. The solution was filtered through 0.45 micron filter and the clear solution was subjected to slow solvent evaporation at 25° C. by covering the tube with paraffin film with a pin hole. After, 2 days, the obtained solid was analyzed by XRD—Form AM1; as shown in FIG. 1.

### Example 2: Preparation of Avasopasem Manganese Form AM2

Avasopasem manganese (0.1 grams) was dissolved in tetrahydrofuran (0.3 ml) and water (0.4 ml) at 25-30° C. in a test tube. The solution was filtered through 0.45 micron filter and the clear solution was subjected to slow solvent evaporation at 25° C. by covering the tube with a paraffin film with a pin hole. After, 2 days, the obtained solid was analyzed by XRD—Form AM2; as shown in FIG. 2.

### Example 3: Preparation of Avasopasem Manganese Form AM3

Avasopasem manganese (AM1, 0.1 grams) was charged in a mortar and pestle; water was added (2-3 drops) and strongly grinded for 2-5 minutes. The obtained solid was analyzed by XRD—Form AM3; as shown in FIG. 3.

### Example 4: Preparation of Avasopasem Manganese Form AM4

Avasopasem manganese (Form AM1, 14.8 grams), 1,2,-Dimethoxyethane (120 ml) and water (30 ml) were charged into a round bottom flask. The obtained suspension mass was heated to 85° C. and maintained at the same temperature under stirring (800 rpm) for 30 minutes. Further, the reaction mass was cooled to 70° C. and slurry was stirred (800 rpm) at 70° C. for 16 hours. Slurry was cooled to 25° C. during 1 hour and filtered under vacuum for 30 min. The obtained solid was analyzed by XRD and designated as Form AM4; as shown in FIG. 4.

### Example 5: Preparation of Avasopasem Manganese Form AM4

Avasopasem manganese (Form AM1, 0.15 grams) and water (0.75 ml) were charged into a glass vial. The obtained suspension mass was heated to 60° C. and maintained at the same temperature under stirring (800 rpm) for 1 day. Slurry mass was filtered under vacuum at 60° C. and kept on suction at 25-30° C. for 5 minutes to dry. The obtained solid was analyzed by XRD-Form AM4.

### Example 6: Preparation of Avasopasem Manganese Form AM5

Avasopasem manganese (0.15 grams) was stirred in aniline (0.5 ml) at 60° C. for 24 hours. Obtained slurry was filtered under vacuum at 60° C. and dried about 10 minutes at 25-30° C. The obtained solid was analyzed by XRD and designated as Form AM5; as shown in FIG. 5.

### Example 7: Preparation of Avasopasem Manganese Form AM6

Avasopasem manganese (Amorphous form, 0.2 grams) was dried at 80° C. for 45 minutes followed by drying at 120° C. for 30 minutes and dried finally at 160° C. for 45 minutes on Radley's hot plate instrument under nitrogen atmosphere. The obtained solid was analyzed by XRD and designated as Form AM6; as shown in FIG. 6.

### Example 8: Preparation of Avasopasem Manganese Form AM6

Avasopasem manganese (Amorphous form, 0.02 grams) was dried at 160° C. for 10 minutes in TGA instrument under nitrogen atmosphere. The obtained solid was analyzed by XRD—Form AM6.

### Example 9: Preparation of Amorphous Avasopasem Manganese

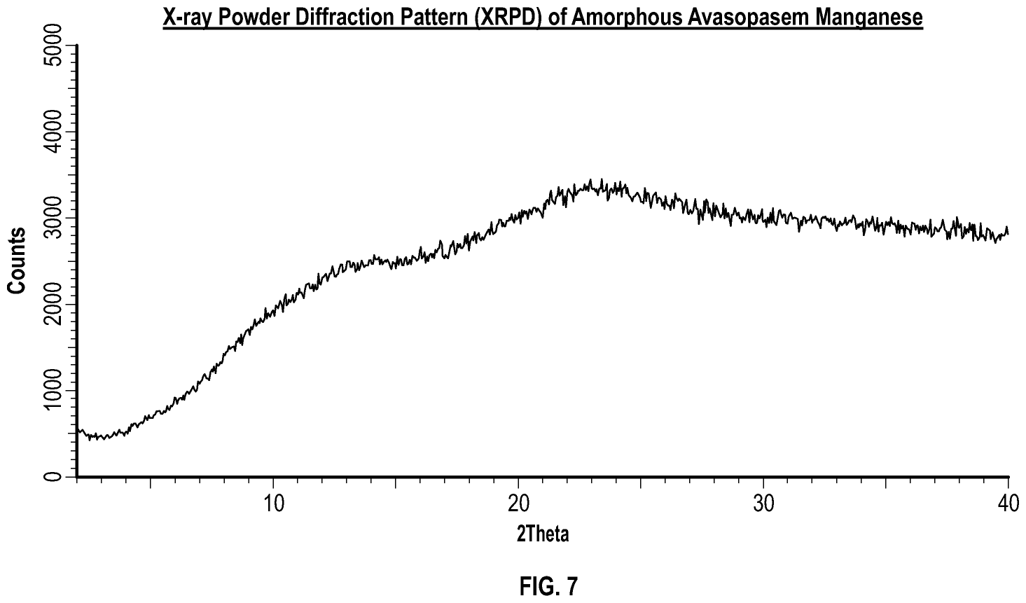

Avasopasem manganese (Form AM1, 1 gram) was dissolved in water (75 ml) at 25° C. The solution was filtered through hyflo bed and the clear solution was frozen by using liquid nitrogen and subjected to lyophilization (condenser temperature: −80° C., vacuum: less than 500 mtorr) for 20 hours. The obtained solid was analyzed by XRD and designated as amorphous form of Avasopasem manganese; as shown in FIG. 7.

### Example 10: Preparation of Avasopasem Manganese Form AM1

Avasopasem Manganese (Form AM2, 0.07 grams) and isopropanol (2 ml) were charged into a test tube. The mixture was heated to 65° C. and the clear solution was then distilled off at 65° C. under vacuum for 1 hr. The obtained solid was dried at 65° C. under vacuum over rotary evaporator for 30 minutes. The obtained solid was analyzed by XRD—Form AM1.

### Example 11: Preparation of Avasopasem Manganese Form AM1

Avasopasem manganese (Form AM4, 0.07 grams) and ethanol (0.35 ml) were charged into a test tube. The mixture was heated to 65° C.; the obtained clear solution was cooled to 25° C. at the rate of 1° C./minute and stirred at 25° C. for 1 day. The obtained solid was filtered under vacuum at 25° C. for 5 minutes. The obtained solid was analyzed by XRD—Form AM1.

### Example 12: Preparation of Avasopasem Manganese Form AM1

Avasopasem Manganese (Form AM2, 0.07 grams) was dissolved in water (2 ml) at 60° C. The clear solution was then distilled off at 60-65° C. under vacuum for 1 hr. The obtained solid was dried at 65° C. under vacuum over rotary evaporator for 30 minutes. The obtained solid was analyzed by XRD—Form AM1.

### Example 13: Preparation of Avasopasem Manganese Form AM1

Avasopasem manganese (Form AM2, 0.07 grams) was dissolved in ethanol (2 ml) at 60-65° C.; the obtained clear solution was cooled to 25° C. and then added methy-tert butyl ether (5 ml) and maintained at 25° C. for about 18 hours. The obtained solid was filtered under vacuum at 25° C. for 5 minutes. The obtained solid was analyzed by XRD—Form AM1.

### Example 14: Preparation of Avasopasem Manganese Form AM2

Avasopasem manganese (Form AM1, 0.15 grams) and water (0.75 ml) were charged into a glass vial. Obtained suspension mass was cooled and stirred at 0-5° C. for 24 hours. Slurry was filtered under vacuum at 0-5° C. and kept on suction at 25-30° C. for 5 minutes to dry. The obtained solid was analyzed by XRD—Form AM2.

### Example 15: Preparation of Avasopasem Manganese Form AM2

Avasopasem Manganese (Form AM1, 0.15 grams) and water (0.75 ml) were charged into a glass vial. Obtained suspension was stirred at 25° C. for 24 hours. Slurry was filtered under vacuum at 25° C. and kept on suction at 25-30° C. for 5 minutes to dry. The obtained solid was analyzed by XRD—Form AM2.

### Example 16: Preparation of Avasopasem Manganese Form AM2

Avasopasem manganese (Form AM4, 0.07 grams) was dissolved in water (1.3 ml) at 60-65° C.; the obtained clear solution was cooled to 25° C. Then added 1, 2-dimethoxyethane (3.9 ml) and maintained at 0-5° C. for about 4 hours. The obtained solid was filtered under vacuum at 25° C. for 5 minutes. The obtained solid was analyzed by XRD—Form AM2.

### Example 17: Preparation of Avasopasem manganese Form AM4

Avasopasem Manganese (Amorphous, 0.10 grams) was weighed in a petri dish and exposed to 90%±5% RH at 60° C.±2° C. for 3 days. The obtained solid was analyzed by XRD-Form AM4.

### Example 18: Preparation of Avasopasem Manganese Form AM4

Avasopasem manganese (form AM1, 0.15 grams) was charged in a reaction vial with septum. A mixture of isopropyl alcohol: water (90:10 v/v ratio, 0.5 ml) was added at 25° C. and slurry mass was maintained under stirring at 60° C. for 1 day. The hot reaction mixture was filtered and dried under vacuum at 25° C. for 10-15 minutes. The obtained solid was analyzed by XRD—Form AM4.

### Example 19: Preparation of Avasopasem Manganese Form AM5

Avasopasem manganese (AM1, 0.15 grams) was stirred in aniline (0.5 ml) suspension at 60° C. for 24 hours. Obtained slurry was filtered under vacuum at 60° C. and dried about 10 minutes at 25-30° C. The obtained solid was analyzed by XRD and determined to be Form AM4.

1. A crystalline form of Avasopasem manganese designated as Form AM1,
characterized by data selected from one or more of the following:
an X-ray powder diffraction pattern substantially as depicted in FIG. 1;
an X-ray powder diffraction pattern having peaks at 7.8, 8.2, 11.2, 14.1 and 23.5 degrees 2-theta±0.2 degrees 2-theta; or
combinations of these data.

2. The crystalline Form AM1 of Avasopasem manganese according to claim 1, wherein said crystalline is characterized by an XRPD having peaks at: 7.8, 8.2, 11.2, 14.1 and 23.5 degrees 2-theta±0.2 degrees 2-theta; and further characterized by having one, two, three, four or five additional peaks selected from 16.5, 18.5, 24.8, 27.7 and 32.1 degrees 2-theta±0.2 degrees 2-theta.

3. The crystalline Form AM1 of Avasopasem manganese according to claim 1, wherein said crystalline Form 1 is characterized by an XRPD having peaks at: 7.8, 8.2, 11.2, 14.1, 16.5, 18.5, 23.5, 24.8, 27.7 and 32.1 degrees 2-theta±0.2 degrees 2-theta.

4. The crystalline Form AM1 of Avasopasem manganese according to claim 1.

5. A crystalline form of Avasopasem manganese designated as Form AM2, characterized by data selected from one or more of the following:
an X-ray powder diffraction pattern substantially as depicted in FIG. 2;
an X-ray powder diffraction pattern having peaks at 6.8, 9.7, 13.0, 22.9 and 23.9 degrees 2-theta±0.2 degrees 2-theta; or
combinations of these data.

6. The crystalline Form AM2 of Avasopasem manganese according to claim 5, wherein said crystalline is characterized by an XRPD having peaks at: 6.8, 9.7, 13.0, 22.9 and 23.9 degrees 2-theta±0.2 degrees 2-theta; and further characterized by having one, two, three, four or five additional peaks selected from 13.3, 15.4, 19.4, 24.3 and 24.9 degrees 2-theta±0.2 degrees 2-theta.

7. The crystalline Form AM2 of Avasopasem manganese according to claim 5, wherein said crystalline Form AM2 is characterized by an XRPD having peaks at: 6.8, 9.7, 13.0, 13.3, 15.4, 19.4, 22.9, 23.9, 24.3 and 24.9 degrees 2-theta±0.2 degrees 2-theta.

8. The crystalline Form AM2 of Avasopasem manganese according to claim 5 which is a hydrate.

9. A crystalline form of Avasopasem manganese designated as Form AM4, characterized by data selected from one or more of the following:
an X-ray powder diffraction pattern substantially as depicted in FIG. 4;
an X-ray powder diffraction pattern having peaks at 7.9, 14.3, 15.8, 25.4 and 28.0 degrees 2-theta±0.2 degrees 2-theta; or
combinations of these data.

10. The crystalline Form AM4 of Avasopasem manganese according to claim 9, wherein said crystalline is characterized by an XRPD having peaks at: 7.9, 14.3, 15.8, 25.4 and 28.0 degrees 2-theta±0.2 degrees 2-theta; and further characterized by having one, two, three, four or five additional peaks selected from 6.7, 9.8, 19.0, 21.5 and 33.8 degrees 2-theta±0.2 degrees 2-theta.

11. The crystalline Form AM4 of Avasopasem manganese according to claim 9, wherein said crystalline Form AM4 is characterized by an XRPD having peaks at: 6.7, 7.9, 9.8, 14.3, 15.8, 19.0, 21.5, 25.4, 28.0 and 33.8 degrees 2-theta±0.2 degrees 2-theta.

12. The crystalline Form AM4 of Avasopasem manganese according to claim 9 which is a hydrate.

13. Crystalline Avasopasem manganese according to claim 1, which contains no more than about 20% of any other crystalline forms of Avasopasem manganese.

14. Crystalline Avasopasem manganese according to claim 1, which contains no more than about 20%, no more than about 10%, no more of amorphous Avasopasem manganese.

15. A pharmaceutical composition comprising a crystalline form according to claim 1 and at least one pharmaceutically acceptable excipient.

16. (canceled)

17. A process for preparing a pharmaceutical composition according to claim 15, comprising combining a crystalline form according to claim 1 with at least one pharmaceutically acceptable excipient.

18. A medicament comprising the crystalline form according to claim 1.

19. (canceled)

20. A method of treating radiation-induced severe oral mucositis comprising administering a therapeutically effective amount of a crystalline product according to claim 1, to a subject in need of the treatment.

21. Crystalline Avasopasem manganese according to claim 5, which contains no more than about 20% of amorphous Avasopasem manganese.

22. A pharmaceutical composition comprising a crystalline form according to claim 5 and at least one pharmaceutically acceptable excipient.

23. A process for preparing a pharmaceutical composition, comprising combining a crystalline form according to claim 5 with at least one pharmaceutically acceptable excipient.

24. A medicament comprising the crystalline form according to claim 5.

25. Crystalline Avasopasem manganese according to claim 9, which contains no more than about 20% of amorphous Avasopasem manganese.

26. A pharmaceutical composition comprising a crystalline form according to claim 9 and at least one pharmaceutically acceptable excipient.

27. A process for preparing a pharmaceutical composition, comprising combining a crystalline form according to claim 9 with at least one pharmaceutically acceptable excipient.

28. A medicament comprising the crystalline form according to claim 9.
